(19) United States
(12) Patent Application Publication
(10) Pub. No.: US 2008/0051661 A1
(43) Pub. Date: Feb. 28, 2008

(54) ULTRASONIC DIAGNOSTIC APPARATUS AND ULTRASONIC DIAGNOSTIC METHOD

(75) Inventors: Muneki KATAGUCHI, Nasushiobara-Shi (JP); Tetsuya KAWAGISHI, Nasushiobara-Shi (JP); Yasuhiko ABE, Otawara-Shi (JP); Shinichi HASHIMOTO, Otawara-Shi (JP)

Correspondence Address: OBLON, SPIVAK, MCCLELLAND MAIER & NEUSTADT, P.C., 1940 DUKE STREET, ALEXANDRIA, VA, 22314, US

(73) Assignees: KABUSHIKI KAISHA TOSHIBA, Tokyo (JP); TOSHIBA MEDICAL SYSTEMS CORPORATION, Otawara-shi (JP)

(21) Appl. No.: 11/844,652

(22) Filed: Aug. 24, 2007

(30) Foreign Application Priority Data
Aug. 28, 2006 (JP) 2006-230863

(51) Int. Cl. A61B 8/00

(52) U.S. Cl. 600/455; 600/441

(57) ABSTRACT

An ultrasonic diagnostic apparatus includes a Doppler velocity information acquiring unit, a region-of-interest-setting unit and an instantaneous flow calculating unit. The Doppler velocity information acquiring unit is configured to acquire three-dimensional Doppler velocity information from an object by a three-dimensional scan with transmission and reception of an ultrasonic wave. The region-of-interest-setting unit is configured to set a region of interest spatially. The instantaneous flow calculating unit is configured to calculate an instantaneous blood flow in the region of interest with using the three-dimensional Doppler velocity information.

### BACKGROUND OF THE INVENTION

1. Field of the Invention

The present invention relates to an ultrasonic diagnostic apparatus and an ultrasonic diagnostic method for obtaining three-dimensional blood flow information by acquiring a Doppler signal from an object, and more particularly, to an ultrasonic diagnostic apparatus and an ultrasonic diagnostic method which display a time variation of blood flow quantity obtained by integrating pieces of velocity information of a blood flow.

2. Description of the Related Art

An ultrasonic diagnostic apparatus is an image diagnostic apparatus which obtains a tomographic image of a tissue in an object by transmitting and receiving an ultrasonic wave into and from the object without invasiveness. A technique of measuring a velocity of a blood flow or a tissue in an object using Doppler effect of an ultrasonic wave among ultrasonic diagnoses is called to an ultrasonic Doppler method. Further, an ultrasonic Doppler method using an ultrasonic pulse is known as a PWD (pulsed wave Doppler method).

Conventionally, a blood flow quantity (hereinafter, simply blood flow) is calculated from a velocity of a blood flow by PWD. In a general method for measuring a blood flow, a velocity of a blood flow is calculated by a two-dimensional scan under PWD.

FIG. 21 is a diagram showing a time variation of blood flow velocity measured by a conventional two-dimensional scan.

In FIG. 21, the abscissa denotes time and the ordinate denotes a blood flow velocity. As shown in FIG. 21, a blood flow velocity varies periodically and shows peaks at constant intervals. A blood flow is calculated from a peak value Vp of the blood flow velocity.

FIG. 22 is a schematic diagram explaining a general way to obtain a blood flow using a blood flow velocity measured by a conventional two-dimensional scan.

As shown in FIG. 22, it is assumed that an instantaneous blood flow velocity distribution shapes an axisymmetric elliptic paraboloid. Then, a two-dimensional instantaneous blood flow can be calculated by using a peak value Vp, of the blood flow velocity, corresponding to the center axis of the velocity distribution shaping the elliptic paraboloid as shown in expression (1).

V_{2D}=Vp×S (1)

wherein
V2D: a two-dimensional instantaneous blood flow (cm³/s);
Vp: a peak value of blood flow velocity on the center axis (cm/s); and
S: a cross section area of the blood flow (cm²).

In recent years, a three-dimensional ultrasonic diagnostic apparatus that can measure a blood flow precisely and simply is designedv (see, for example, Japanese Patent Application (Laid-Open) No. 2000-201930). A blood flow is calculated from color Doppler blood flow velocity information acquired by a three-dimensional scan in this three-dimensional ultrasonic diagnostic apparatus. Highly precise and stable results can be obtained by a method for calculating a blood flow from three-dimensional blood flow velocity information, compared with a method of estimating a volume with processing such as interpolation from a two-dimensional tomographic image. Finally, a value of the blood flow obtained by the above method is displayed on a monitor numerically.

Moreover, a technique that measures a cardiac output by integrating automatically color Doppler velocity information acquired by a three-dimensional scan is also designed.

However, according to the three-dimensional color Doppler method by the conventional ultrasonic diagnostic apparatus, only component in a sound axis direction, which is a travel direction of an ultrasonic wave, among the velocity information on the acquired blood flow is detected. For this reason, there is a problem that detection accuracy of a blood flow velocity falls extremely when an angle between a direction of a blood flow and a sound axis of an ultrasonic wave becomes near 90 degrees. Consequently, there is a possibility that a blood flow velocity may be recognized incorrectly.

Therefore, it may be difficult to calculate a blood flow correctly, depending on a method of scanning, i.e., relative inclination between a direction of a blood flow and a sound axis of an ultrasonic wave, even if a method of calculating a blood flow precisely from velocity information on a three-dimensional blood flow is adopted.

In order to solve this problem, a scan must be carried out so that a relative angle between a direction of a blood flow and a sound axis of an ultrasonic wave may turn into the smallest possible angle. However, a scan of an ultrasonic diagnosis may be carried out only from the restricted position of an object. For example, bones and lungs must be avoided when scanning since they do not pass an ultrasonic wave.

Corresponding to the above, as a method for raising accuracy of measuring blood flow velocities as much as possible in the state where an angle between a direction of a blood flow and a sound axis of an ultrasonic wave is large, it is known that the method of exploring a position where a detected velocity of a blood flow and a absolute value of blood flow obtained by calculating based on a velocity of a blood flow become maximum with moving a scanning position.

In order to find a scanning position where the maximum blood flow is obtained while indicating the blood flow numerically acquired by the conventional three-dimensional scan, a step is needed to maximize the numerical indication of the blood flow by changing a scanning position. For this reason, an operator must gaze steadily at an ultrasonic image and the numerically indicated blood flow simultaneously. In addition, the operator must memorize the scanning position where the maximum blood flow was detected. Therefore, realistically, it is in a difficult situation to capture the maximum flow velocity and the maximum blood flow easily because of such a burden occurring.

Even in regarding the cardiac output, it changes depending on a scanning position by an ultrasonic wave. This is based on the scanning angle dependability peculiar to the ultrasonic Doppler method, as mentioned above. Therefore, it is difficult to judge whether the cardiac output was measured in suitable accuracy when the cardiac output is measured by integrating with the blood flow of a certain scanning position in the direction of time.

### SUMMARY OF THE INVENTION

The present invention has been made in light of the conventional situations, and it is an object of the present invention to provide an ultrasonic diagnostic apparatus and an ultrasonic diagnostic method which can measure a blood flow at a more appropriate region.

The present invention provides an ultrasonic diagnostic apparatus comprising: a Doppler velocity information acquiring unit configured to acquire three-dimensional Doppler velocity information from an object by a three-dimensional scan with transmission and reception of an ultrasonic wave; a region-of-interest-setting unit configured to set a region of interest spatially; and an instantaneous flow calculating unit configured to calculate an instantaneous blood flow in the region of interest with using the three-dimensional Doppler velocity information, in an aspect to achieve the object.

The present invention also provides an ultrasonic diagnostic method comprising steps of: acquiring three-dimensional Doppler velocity information from an object by a three-dimensional scan with transmission and reception of an ultrasonic wave; setting a region of interest spatially; and calculating an instantaneous blood flow in the region of interest with using the three-dimensional Doppler velocity information, in an aspect to achieve the object.

The ultrasonic diagnostic apparatus and the ultrasonic diagnostic method as described above make it possible to measure a blood flow at a more appropriate region.

### BRIEF DESCRIPTION OF THE DRAWINGS

In the accompanying drawings:

FIG. 1 is a functional block diagram showing an ultrasonic diagnostic apparatus according to an embodiment of the present invention;

FIG. 2 is a flowchart showing a procedure for obtaining and displaying a temporal variation of blood flow together with an ultrasonic image by the ultrasonic diagnostic apparatus 1 shown in FIG. 1;

FIG. 3 is a schematic diagram showing an example of three-dimensional color Doppler velocity information calculated by the color Doppler calculation unit shown in FIG. 1;

FIG. 4 is a diagram showing an example of cross sections which are targets of a three-dimensional scan by the ultrasonic diagnostic apparatus 1 shown in FIG. 1;

FIG. 5 is an overhead view of the cross sections shown in FIG. 4;

FIG. 6 is a diagram showing an example of designing sizes of spaces between cross sections to be a scan target in conformity to a ROI by the ultrasonic diagnostic apparatus shown in FIG. 1;

FIG. 7 is an overhead view of the cross sections shown in FIG. 6;

FIG. 8 is a diagram showing an example of temporal variation graph of blood flow displayed by using a moving bar on the display unit shown in FIG. 1;

FIG. 9 is a diagram showing an example of temporal variation graph of blood flow displayed by a scroll method on the display unit shown in FIG. 1;

FIG. 10 is a diagram showing an example of plural ROIs set by the ROI inputting unit shown in FIG. 1;

FIG. 11 is a diagram showing an example of graphs respectively indicating temporal variations of blood flows in the respective ROIs shown in FIG. 10 to be displayed;

FIG. 12 is a diagram showing an example of temporal variation graph of blood flow obtained in case of performing a scan with moving the 2D arrayed probe shown in FIG. 1;

FIG. 13 is a diagram explaining a way to calculate a cardiac output based on a temporal variation graph of blood flow in the cardiac output calculation unit shown in FIG. 1;

FIG. 14 is an enlarged view showing the temporal variation of blood flow between times selected on the temporal variation graph of blood flow shown in FIG. 13;

FIG. 15 is an electrocardiogram showing a normal stable ECG waveform normally used for detection of one cardiac cycle;

FIG. 16 is an example of electrocardiogram showing a disturbed ECG waveform due to changing a body position of object;

FIG. 17 is a diagram showing an example of display of a region for instantaneous blood flow used for calculating a cardiac output with using a marker shown by a dotted line on the display unit shown in FIG. 1;

FIG. 18 is a diagram showing an example of display of a region for instantaneous blood flow used for calculating a cardiac output with using markers each shown by a triangular symbol on the display unit shown in FIG. 1;

FIG. 19 is a diagram showing an example of display of a region for instantaneous blood flow used for calculating a cardiac output by color or pattern indication on the display unit shown in FIG. 1;

FIG. 20 is a diagram showing an example of window in case of displaying a three-dimensional color Doppler image, a graph indicating a temporal variation of blood flow and a cardiac output together on the display unit of the ultrasonic diagnostic apparatus shown in FIG. 1;

FIG. 21 is a diagram showing a time variation of blood flow velocity measured by a conventional two-dimensional scan; and

FIG. 22 is a schematic diagram explaining a general way to obtain a blood flow using a blood flow velocity measured by a conventional two-dimensional scan.

### DESCRIPTION OF THE PREFERRED EMBODIMENTS

An ultrasonic diagnostic apparatus and an ultrasonic diagnostic method according to embodiments of the present invention will be described with reference to the accompanying drawings.

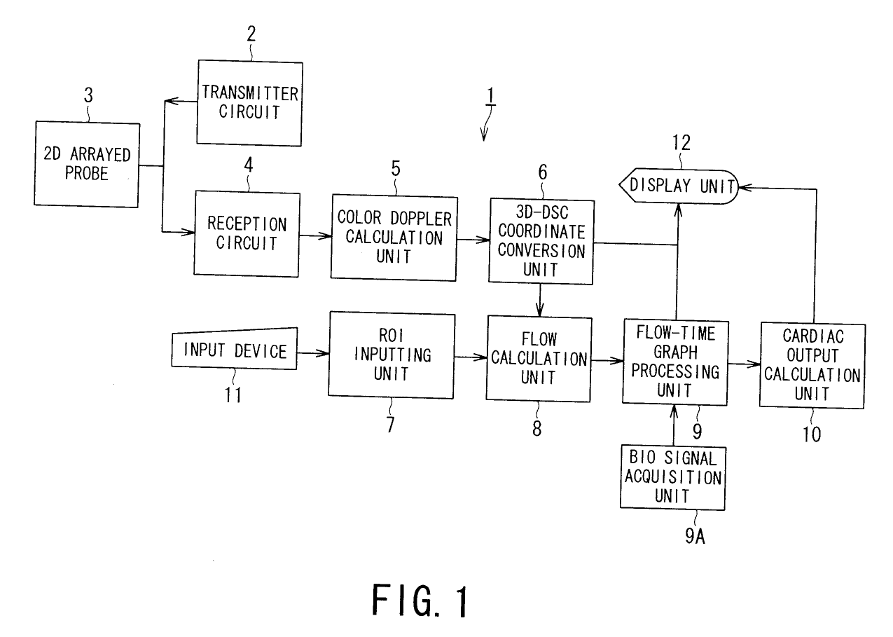

FIG. 1 is a functional block diagram showing an ultrasonic diagnostic apparatus according to an embodiment of the present invention.

An ultrasonic diagnostic apparatus 1 includes a transmitter circuit, a 2D (two-dimensional) arrayed probe 3, a reception circuit 4, a color Doppler calculation unit 5, 3D-DSC (three-dimensional-digital scan converter) coordinate conversion unit 6, an ROI (region of interest) inputting unit 7, a flow calculation unit 8, a flow-time graph processing unit 9, a cardiac output calculation unit 10, an input device 11 and a display unit 12. Each element can be configured by a circuit or a computer in which program is installed.

The transmitter circuit 2 has functions to generate a pulse signal as a transmission signal so that an ultrasonic wave may be transmitted towards a desired direction at a desired transmitting timing and transmitting intervals from the 2D arrayed probe 3, and to apply the generated transmission signal to the 2D arrayed probe 3.

The 2D arrayed probe 3 is provided with a plurality of ultrasonic transducers for transmitting and receiving an ultrasonic wave. Each ultrasonic transducer is arranged in two dimensions. Further, the 2D arrayed probe 3 is configured so that it enables the three-dimensional scan by electronic scan carried out by controlling delay times with the respective ultrasonic transducers. Further more, the 2D arrayed probe 3 transmits a transmission signal given as an electric signal from the transmitter circuit 2 into an object as an ultrasonic wave, while it is configured so that it receives an ultrasonic wave echo generated within the object to be converted into an echo signal as an electric signal, and provides the echo signal to the reception circuit 4. Especially, besides echo signals for a B mode image which is an ultrasonic tomographic image, three-dimensional Doppler signals for generating a blood-flow image by the ultrasonic Doppler method are received by the 2D arrayed probe 3, and the received Doppler signals are outputted to the reception circuit 4.

The reception circuit 4 has a function to acquire Doppler signals and echo signals for a B mode image from the 2D arrayed probe 3, provide the echo signals for the B mode image to a B mode image-processing system (not shown) and provide the Doppler signals to the color Doppler calculation unit 5.

The color Doppler calculation unit 5 has a function to calculate color Doppler velocity information which is three-dimensional velocity information of a blood flow from Doppler signals acquired from the reception circuit 4, and to provide the calculated color Doppler velocity information to the 3D-DSC coordinate conversion unit 6.

The 3D-DSC coordinate conversion unit 6 has a function to perform coordinate conversion processing for converting a scan type of color Doppler velocity information acquired from the color Doppler calculation unit 5 from a scan mode by the 2D arrayed probe 3 into a television scan mode, and has a function to provide the color Doppler velocity information after the coordinate conversion to thea display unit 12 and the flow calculation unit 8. Further, the 3D-DSC coordinate conversion unit 6 is equipped with necessary image-processing functions, such as freezing and interpolation processing.

That is, a color Doppler image is displayed on the display unit 12 by outputting the color Doppler velocity information in the television scan mode from the 3D-DSC coordinate conversion unit 6 to the display unit 12. Furthermore, in the case of B mode image information generated in the B mode image-processing system (not shown) is provided to the display unit 12, the color Doppler image may be superpositioned on a B mode image for displaying on the display unit 12.

The ROI inputting unit 7 has a function to set an ROI according to directional information from the input device 11, and has a function to provide the ROI that has been set to the flow calculation unit 8. An ROI may be spatially set as a two-dimensional area on an arbitrary plane or curved surface.

The flow calculation unit 8 has a function to calculate a instantaneous blood flow in an ROI acquired from the ROI inputting unit 7 based on three-dimensional color Doppler velocity information acquired from the 3D-DSC coordinate conversion unit 6, and has a function to provide the instantaneous blood flow acquired by the calculation to the flow-time graph processing unit 9. Moreover, when the color Doppler velocity information for calculating the instantaneous blood flow in the ROI is insufficient, the flow calculation unit 8 is configured so that it enables to obtain the insufficient color Doppler velocity information by interpolation from other acquired color Doppler velocity information.

The flow-time graph processing unit 9 has a function to make graph information for displaying a graph showing a time change of the blood flow on the display unit 12, based on a value of the instantaneous blood flow at each time in the ROI acquired from the flow calculation unit 8, and a function to allow a graph to be displayed on the display unit 12 by providing the generated graph information to the display unit 12. Further, the flow-time graph processing unit 9 is configured to provide the generated graph information to the cardiac output calculation unit 10.

Moreover, a bio signal acquisition unit 9A may be provided in the flow-time graph processing unit 9. The bio signal acquisition unit 9a has a function to acquire a signal that shows time changes of a desired bio signal from an object. The bio signal acquisition unit 9A may be composed of an ECG (electro cardiogram) signal acquisition unit, which acquires an ECG signal from an object.

Furthermore, when a bio signal, such as an ECG signal, is acquired, the flow-time graph processing unit 9 is configured to make graph information as the time changes of the bio signal are displayed in parallel on a graph by synchronizing time change of a blood flow with a time phase.

The cardiac output calculation unit 10 has a function to calculate a pumping quantity for 1 beat in a heart, i.e. a cardiac output, based on graph information acquired from the flow-time graph processing unit 9, and a function to provide the calculated cardiac output to the display unit 12 so that the calculated cardiac output is displayed on the display unit 12.

Next, the operation and action of the ultrasonic diagnostic apparatus 1 will be described.

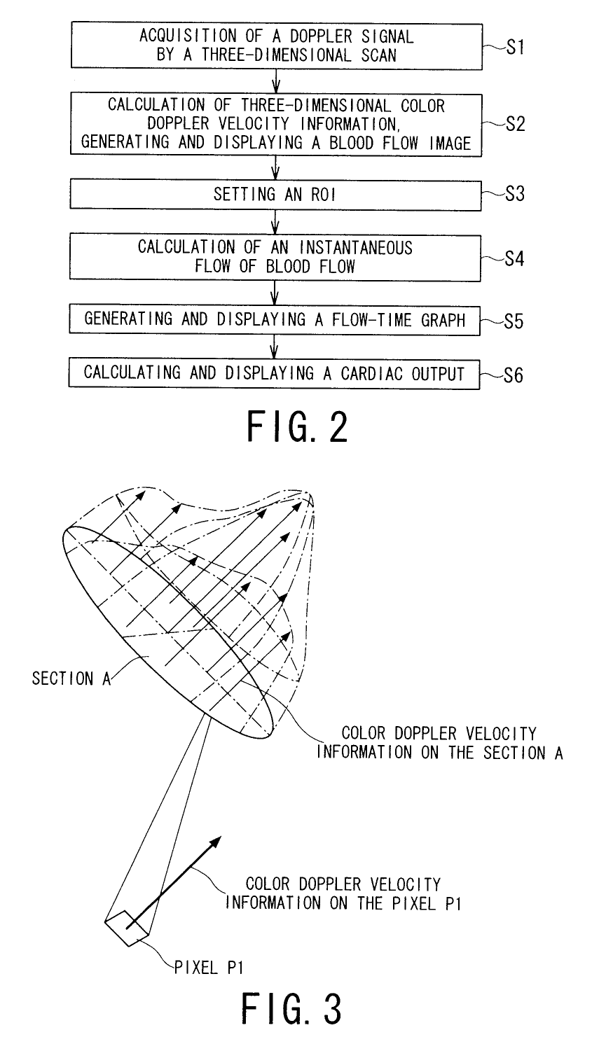

FIG. 2 is a flowchart showing a procedure for obtaining and displaying a temporal variation of blood flow together with an ultrasonic image by the ultrasonic diagnostic apparatus 1 shown in FIG. 1. Reference numerals having numbers added to reference symbol S denote steps in the flowchart shown in FIG. 2.

First, in Step S1, Doppler signals from an object are acquired by a three-dimensional scan. That is, the transmitter circuit 2 generates a pulse signal as a transmission signal, and applies the generated transmission signal to the 2D arrayed probe 3. Then, the 2D arrayed probe 3 transforms the transmission signal, which is an electric signal, into an ultrasonic wave, and transmits the ultrasonic wave to a position on a predetermined depth in the object along with a scanning line. Finally, the 2D arrayed probe 3 receives Doppler signals generated in the object, and the received Doppler signals are transformed into the electric signals before outputted to the reception circuit 4. The reception circuit 4 provides the Doppler signals received from the 2D arrayed probe 3 to the color Doppler calculation unit 5.

Such acquisition of Doppler signals is performed in three dimensions by the three-dimensional scan. One unit of data for an ultrasonic image obtained by the three-dimensional scan is called ‘Volume’. In order to obtain one Volume, the 2D arrayed probe 3 is directed towards a part where is a generation target of a blood-flow image, transmission and reception of ultrasonic signals on a same scanning line are performed for necessary times, and a scan is performed a plurality of times. Then, pluralities of Doppler signals from each position of the three-dimensional space in the object are accumulated in the color Doppler calculation unit 5.

Next, in Step S2, three-dimensional color Doppler velocity information is obtained from the acquired Doppler signals in the color Doppler calculation unit 5. The color Doppler velocity information has a unit of a certain spatial size at each position of three-dimensional space, and the size, which serves as a unit at each position of the color Doppler velocity information, is called a ‘pixel’ In general, all the pixels required for displaying a blood-flow image is treated as a uniform size.

FIG. 3 is a schematic diagram showing an example of three-dimensional color Doppler velocity information calculated by the color Doppler calculation unit 5 shown in FIG. 1.

As shown in FIG. 3, plural pixels each having a same size are formed in the respective positions on a certain section A. Then, the three-dimensional color Doppler velocity information having a size and direction for every pixel is obtained based on a plurality of Doppler signals on a same scanning line in the color Doppler calculation unit 5.

Finally, the pieces of color Doppler velocity information obtained for every pixel are arranged in the corresponding positions in the three-dimensional space respectively.

Next, the three-dimensional color Doppler velocity information obtained in the color Doppler calculation unit 5 is provided to the 3D-DSC coordinate conversion unit 6. In the 3D-DSC coordinate conversion unit 6, a scan mode of the color Doppler velocity information acquired from the color Doppler calculation unit 5 is converted into a television scan mode from a scan mode by the 2D arrayed probe 3. As a result, the color Doppler velocity information after the coordinate conversion is generated as blood-flow image data for displaying a blood-flow image on the display unit 12. Further, in the 3D-DSC coordinate conversion unit 6, required image processing, such as freezing and interpolation processing, is performed.

The color Doppler velocity information after the coordinate conversion generated in the 3D-DSC coordinate conversion unit 6 is provided to the flow calculation unit 8 and the display unit 12. Thereby, a color Doppler image is displayed on the display unit 12. When B mode image information generated in the B mode image-processing system (not shown) is provided to the display unit 12, the color Doppler image can be superpositioned on the B mode image for displaying on the display unit 12.

The color Doppler image is, as mentioned above, generated from Doppler signals for 1 Volume. Time required in order to complete construction of a color Doppler image of 1 Volume is determined by conditions, such as a sound wave velocity of an ultrasonic wave in an object, an interval of transmitting time of an ultrasonic signal, and a number of scanning lines (a number of times of transmission of an ultrasonic wave) for acquireing three-dimensional Doppler signals for 1 Volume. A number of volumes, which can be constructed in 1 second, determined under these conditions is called a ‘Volume Rate’. The unit of the Volume Rate is ‘Volume/Second’.

Further, repeated construction of a color Doppler image for each Volume makes timely continuous images. Furthermore, the so-called real-time display of the images is realized by displaying the created images on the display unit 12 consecutively.

Next, in Step S3, directional information is inputted into the ROI inputting unit 7 by operating the input device 11, and, a desired region is set for calculating a blood flow as an ROI in the ROI inputting unit 7. At this time, a window (screen) for setting up an ROI is displayed on the display unit 12. The window information for setting up an ROI can be made in the ROI inputting unit 7. Further, an ROI can be easily set by operating the input device 11, such as a mouse, with referring to a window displayed on the display unit 12 by GUI (Graphical User Interface) technology.

Three-dimensional images including a volume rendering image, a surface rendering image, and a single or multiple MPR (multi-planar reconstruction) images may be displayed on the window for setting up an ROI. The three-dimensional image for the ROI setting can be made from the volume data for an ultrasonic image obtained by the three-dimensional scan.

Moreover, a two-dimensional area in a circle centering on a specified point can be set as an ROI by specifying a point on an arbitrary position on a three-dimensional image together with inputting a radius with operation of the input device 11, such as a mouse, for example. However, a two-dimensional area may be made not only into a circle but also into an arbitrary shape. Operation of the input device 11 can determine arbitrarily a plane or a curved surface in the three-dimensional space for preparing a two-dimensional area.

Further, it is possible to expand or reduce the once made ROI to an arbitrary scale by operation of the input device 11, and parallel movement of the ROI in an arbitrary direction and rotation movement of the ROI centering on an axis chosen arbitrarily can be also performed. Furthermore, the ROI may be set not only one, but also more than one. The total range where data acquisition is performed may also be set as the ROI.

Finally, the ROI inputting unit 7 provides the finally set ROI to the flow calculation unit 8.

Next, in Step S4, an instantaneous blood flow is calculated from color Doppler velocity information in the set ROI by the flow calculation unit 8. That is, the flow calculation unit 8 integrates the color Doppler velocity information acquired by the three-dimensional scan for every Volume, and multiplies the size of color pixel to the result of integration. Thereby, the three-dimensional instantaneous blood flow is obtained. In case of obtaining the instantaneous blood flow from the three-dimensional color Doppler velocity information on the section A shown in FIG. 3, the calculation shown in expression (2) is carried out in the flow calculation unit 8.

$${V}_{3 D} = {\sum }^{sectionA} ( Vp \times Sp ) \qquad ( 2 )$$

wherein
V_{3D}: a three-dimensional instantaneous blood flow passing through the cross section A (cm³/s);
Vp: color Doppler velocity information on a pixel (cm/s); and
Sp: a size of pixel (cm²)

Thus, if an instantaneous blood flow is calculated from color Doppler velocity information acquired by a three-dimensional scan, the instantaneous blood flow can be obtained with high accuracy compared with the case where the instantaneous blood flow is calculated from color Doppler velocity information acquired by a two-dimensional scan. The reason is as stated below.

A general blood flow in an object shows a complicated velocity distribution since blood vessels travel in various directions. For this reason, it is difficult to calculate an exact blood flow with a simple calculation using color Doppler velocity information acquired by a two-dimensional scan as shown in expression (1). That is, velocity distribution of a usual blood flow does not become one symmetrical with regard to a central rotation line as shown in FIG. 22. For this reason, there is a limit on accuracy in a measurement of a blood flow by a two-dimensional scan.

On the other hand, it is possible to obtain a complicated velocity distribution of a blood flow three-dimensionally and more appropriately in a measurement of a blood flow by a three-dimensional scan even if blood vessels are traveling in various directions. For this reason, a measurement of a blood flow by a three-dimensional scan is advantageous in accuracy compared with that by a two-dimensional scan.

An instantaneous blood flow obtained from three-dimensional color Doppler velocity information is calculated for every Volume. Therefore, instantaneous blood flows are sequentially calculated from the three-dimensional color Doppler velocity information to continuous Volumes, respectively.

At this point, instantaneous blood flows can be also calculated from color Doppler velocity information on a plurality of sections that are different from a section where an ROI is set.

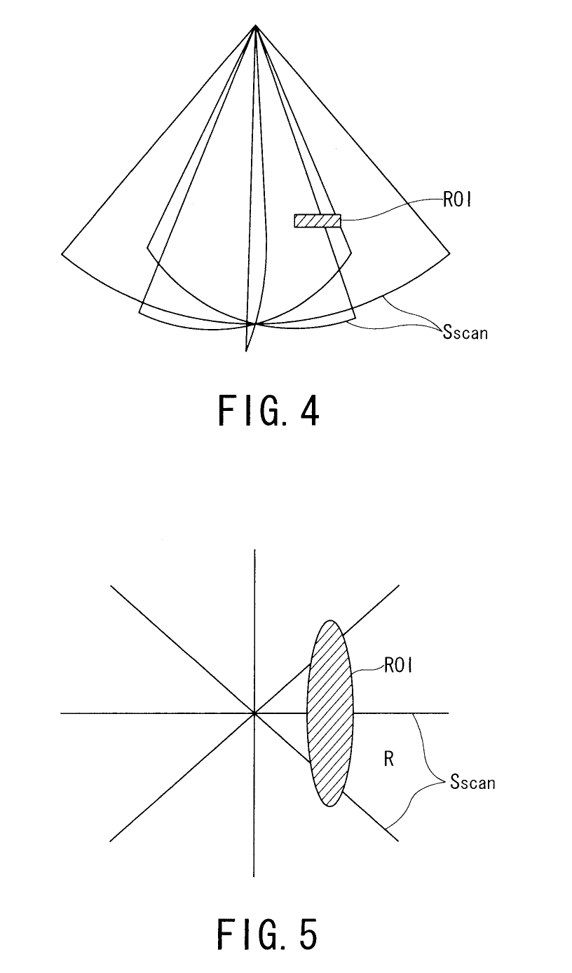

FIG. 4 is a diagram showing an example of cross sections which are targets of a three-dimensional scan by the ultrasonic diagnostic apparatus 1 shown in FIG. 1. FIG. 5 is an overhead view of the cross sections shown in FIG. 4.

As shown in FIG. 4, a plurality of sections Sscan lying in directions perpendicular to a section to which an ROI is set can be set as scanning targets. In such a case, there is a possibility that a region with insufficient data of color Doppler velocity information between the sections Sscan used as the scan targets may occur. Therefore, in case color Doppler velocity information is insufficient for calculating an instantaneous blood flow, the insufficient color Doppler velocity information is estimated with interpolation from other acquirable color Doppler velocity information by the flow calculation unit 8.

When estimating color Doppler velocity information by interpolation, it is necessary to make each space R between the sections Sscan serving as the scan targets small enough in order to secure estimation accuracy. Then, a function for obtaining scan sections Sscan required in order to acquire enough interpolation accuracy for color Doppler velocity information may be provided with the flow calculation unit 8. Further, it is possible to control transmitting conditions of an ultrasonic wave by providing the scan sections Sscan obtained by the flow calculation unit 8 to the transmitter circuit 2.

Furthermore, when providing no function for obtaining scan sections Sscan to the flow calculation unit 8, sections Sscan serving as a scan target by transmission and reception an ultrasonic wave beforehand by an operator so as to obtain color Doppler velocity information with sufficient interpolation accuracy are set.

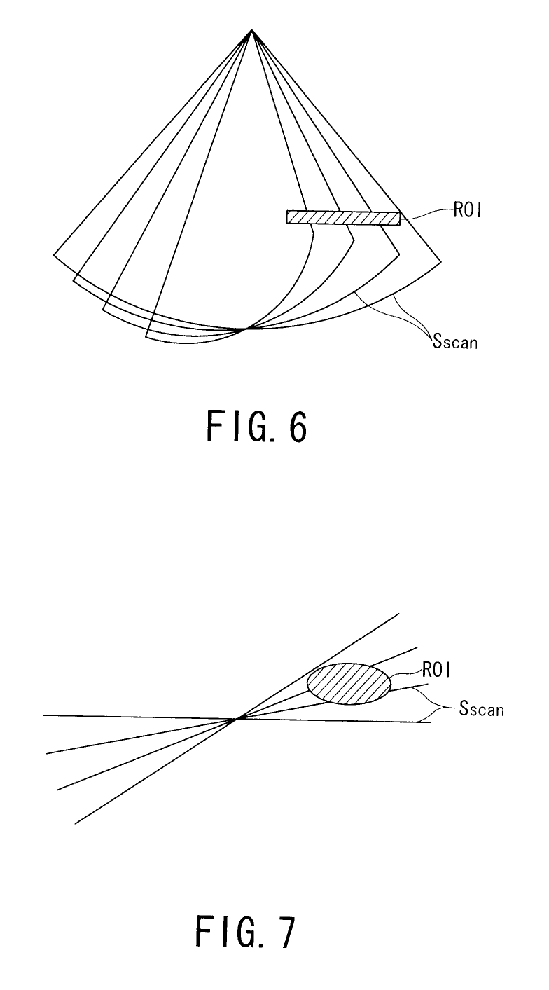

FIG. 6 is a diagram showing an example of designing sizes of spaces between cross sections to be a scan target in conformity to a ROI by the ultrasonic diagnostic apparatus 1 shown in FIG. 1. FIG. 7 is an overhead view of the cross sections shown in FIG. 6.

As shown in FIG. 6, in an area where an ROI is set, when scan sections Sscan is set so that distances between a plurality of sections Sscan serving as a scan target become small locally, the interpolation accuracy of color Doppler velocity information can be secured while suppressing the increase in the number of scanning lines.

Finally, the instantaneous blood flow for every Volume obtained with interpolation processing as occasion demands in this way is provided to the flow-time graph processing unit 9 from the flow calculation unit 8.

Next, in Step S5, the flow-time graph processing unit 9 makes graph information showing time changes of the blood flow from the value of the instantaneous blood flow in the ROI acquired from the flow calculation unit 8. That is, the flow-time graph processing unit 9 plots the time changes of the instantaneous blood flow for every Volume consecutively acquired from the flow calculation unit 8. Thereby, a graph having axes of the instantaneous blood flow and the time can be made. Finally, the flow-time graph processing unit 9 allows the graph to be displayed by providing the made graph information to the display unit 12.

The flowing amount value on the graph is obtained every time the instantaneous blood flow is calculated from the three-dimensional color Doppler velocity information. That is, a blood flow can be calculated for every Volume similar to a color Doppler image. Therefore, it is possible to update the time change graph of the blood flow continuously for every Volume. For this reason, when the time change graph of the blood flow is displayed on the display unit 12 with updating every Volume, real-time display of the graph can be performed similar to a color Doppler image.

The graph may be displayed on the display unit 12 and updated by arbitrary methods.

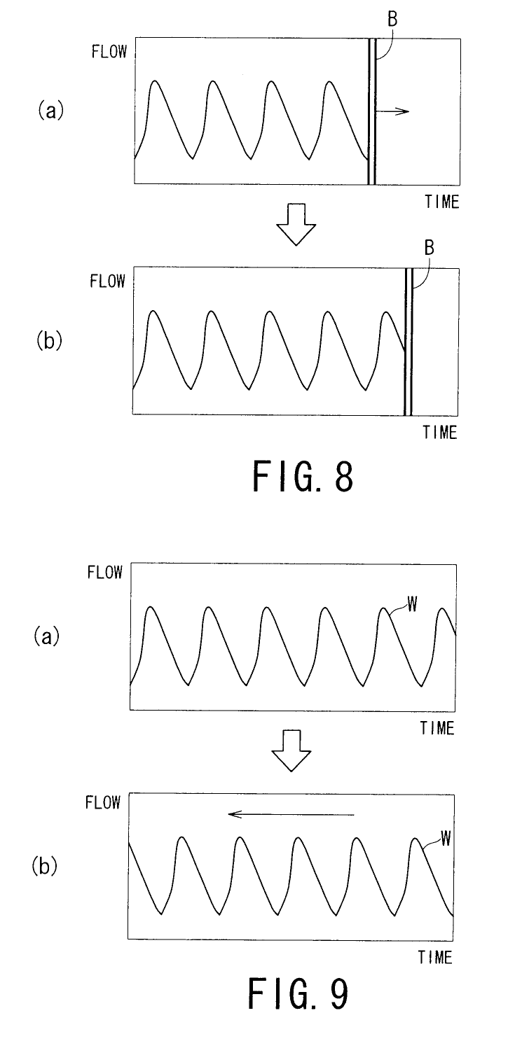

FIG. 8 is a diagram showing an example of temporal variation graph of blood flow displayed by using a moving bar on the display unit 12 shown in FIG. 1.

In (a) and (b) of FIG. 8, the abscissa denotes time and the ordinate denotes a blood flow (quantity). As shown in FIGS. 8(a) and (b), for example, the time change of the blood flow can be indicated by using a moving bar (moving cursor) B which moves in the direction of time. That is, as shown in FIG. 8(a), the time change of the blood flow before a certain time indicated by the moving bar B is displayed. Then, when time passes, as shown in FIG. 8(b), the moving bar B will move in the direction of time, and an additional indication of the time change of the blood flow will be displayed consecutively.

That is, a moving bar method shows a renewal position of an image with the moving cursor. The moving bar method for displaying the graph is a displaying method generally used in the ultrasonic diagnostic apparatus 1, and is often used for displaying an M-mode image or an image obtained by pulse Doppler mode. For this reason, it may be said that it is suitable for interpretation by operators.

FIG. 9 is a diagram showing an example of temporal variation graph of blood flow displayed by a scroll method on the display unit 12 shown in FIG. 1.

In (a) and (b) of FIG. 9, the abscissa denotes time and the ordinate denotes a blood flow (quantity). As shown in FIGS. 9(a) and (b), the time change of the blood flow may also be displayed with a scroll method that moves a waveform W, which shows the amount change of the blood flow, together with time. That is, as shown in FIG. 9(a), the time change of the blood flow before a certain time is displayed on the display unit 12. Then, when time passes, as shown in FIG. 9(b), the waveform W which shows the amount change of the blood flow will move toward left-hand side (the direction of the past), and the time change of the blood flow before the time after the progress of time will be displayed on the display unit 12. That is, by the scroll method, a fixed indication of the newest blood flow is always displayed at the left end.

When plural of ROIs are set, the time change of the blood flow for every ROI is displayed as a graph on the display unit 12.

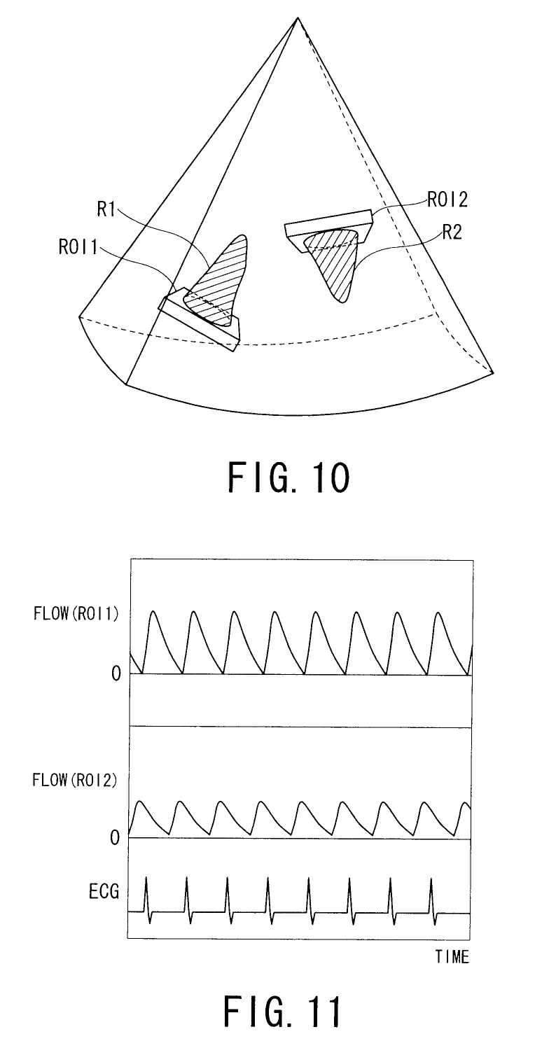

FIG. 10 is a diagram showing an example of plural ROIs set by the ROI inputting unit 7 shown in FIG. 1. FIG. 11 is a diagram showing an example of graphs respectively indicating temporal variations of blood flows in the respective ROIs shown in FIG. 10 to be displayed.

As shown in FIG. 10, two sections, for example, can be set as ROI 1 and ROI 2, respectively, so that each part of regions R1 and R2 where blood flows in the respective scanning ranges exist is contained.

Finally, as shown in FIG. 11, the time change graph of the blood flow in each pf the ROI 1 and ROI 2 shown in FIG. 10 may be displayed in parallel in synchronized with an ECG waveform in time. That is, in FIG. 11, the abscissa denotes time. The ordinate in the upper row in FIG. 11 denotes the blood flow in the ROI 1, the ordinate in the middle row denotes the blood flow in the ROI 2, and the ordinate in the bottom row denotes the value of the ECG waveform, respectively.

Thus, using only velocity information in a certain limited space among color Doppler velocity information acquired by a three-dimensional scan for obtaining an instantaneous flow velocity of a blood flow is useful to diagnosis. For this reason, enabling an operator to designate setting an ROI can raise the operators convenience.

Although an example of a time change graph of a blood flow in case of fixing the 2D arrayed probe 3 was shown so far, it is necessary to find a scanning position where the greatest amount of a blood flow is obtained with moving a position scanned by the 2D arrayed probe 3 in order to raise measurement accuracy of a blood flow velocity when an angle between a flowing direction of the blood flow and a sound axis of an ultrasonic wave is large, as mentioned above. When a scan is carried out with moving the 2D arrayed probe 3, an instantaneous blood flow does not become repetition of a waveform having similar amplitude, but the amplitude will change greatly.

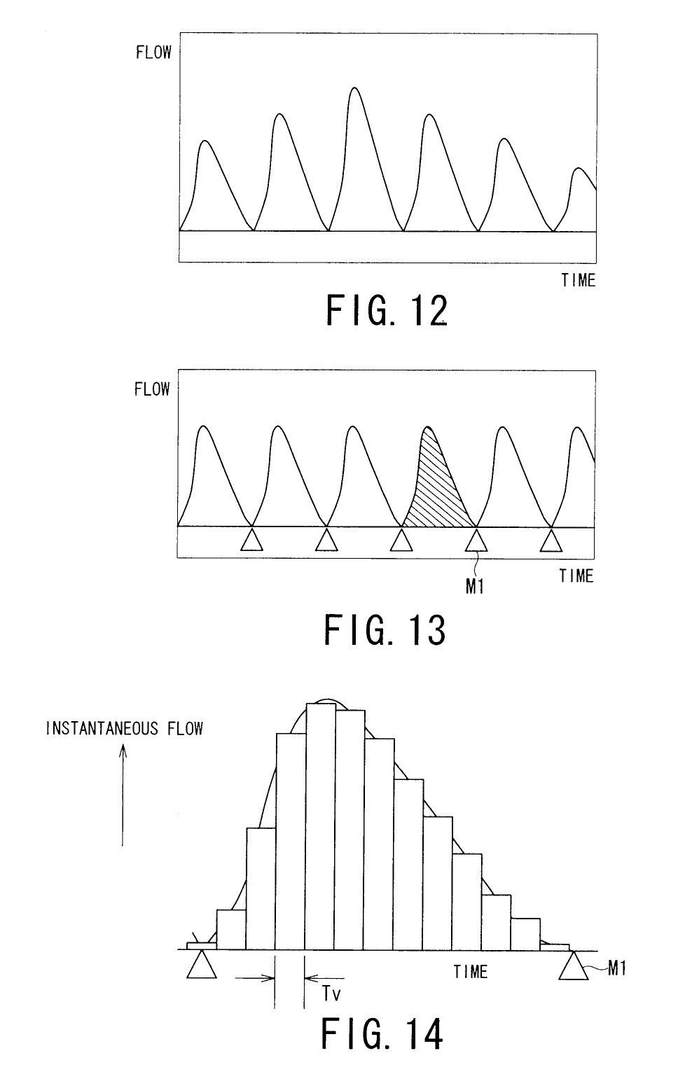

FIG. 12 is a diagram showing an example of temporal variation graph of blood flow obtained in case of performing a scan with moving the 2D arrayed probe 3 shown in FIG. 1.

In FIG. 12, the abscissa denotes time and the ordinate denotes a blood flow (quantity). As shown in FIG. 12, because of a change in an angle between a direction of a blood flow and a sound axis of an ultrasonic wave, the blood flow quantity changes when a scan is carried out with moving the 2D arrayed probe 3. Then, an operator need to perform an operation to grasp a scanning position where a quantity of the blood flow becomes largest.

In such operation, it becomes possible for the operator to grasp the largest value and/or ups and downs of the blood flow easily by enabling the operator to refer to the time change graph of the blood flow as shows in FIG. 12. Since there is a correlation between time and a scan position, the value of the blood flow for every scan position is indirectly recordable as a graph, if the relation between time and the scan position is known. For this reason, the operator can grasp the relation between the value of the blood flow and the scan position not only during the scan but also afterwards. Therefore, it becomes possible for the operator to gaze at an ultrasonic image preponderantly during the scan. Further, if time and the scan position are set beforehand, the operator does not need to memorize the scan position or the blood flow corresponding to the scan position. Furthermore, acquiring an appropriate scan position and the blood flow is expectable by such lightening operator's burden.

The graph information made in this way is also provided to the cardiac output calculation unit 10 from the flow-time graph processing unit 9. Then, if the 2D arrayed probe 3 is being fixed, a cardiac output can be calculated in the cardiac output calculation unit 10.

That is, in Step S6, a cardiac output is calculated from the graph information, which shows the time change of the blood flow in the cardiac output calculation unit 10. The cardiac output is the total flow amount for 1 heart beat equivalent to pumping amount for 1 heart beat in a heart, and is used for diagnosis of heart function. Generally, a blood flow in an object is moving periodically because of heart beat. For this reason, color Doppler velocity information and an instantaneous blood flow acquired from a Doppler signal are changing periodically synchronizing with a heart beat.

Then, after obtaining a period for 1 heart beat using a time change of an instantaneous blood flow, a cardiac output can be calculated from a blood flow in a section of the obtained period.

FIG. 13 is a diagram explaining a way to calculate a cardiac output based on a temporal variation graph of blood flow in the cardiac output calculation unit 10 shown in FIG. 1. FIG. 14 is an enlarged view showing the temporal variation of blood flow between times selected on the temporal variation graph of blood flow shown in FIG. 13.

In FIG. 13, the abscissa denotes time and the ordinate denotes a blood flow (quantity). The cardiac output calculation unit 10 automatically detects a time when the blood flow becomes minimum amount, or a time when the blood flow becomes maximum amount, from a time change graph of a blood flow as shown in FIG. 13. Use of a well-known algorithm generally used for processing such as automatic tracing of pulse Doppler, for example, as the method may be mentioned for a method for detecting the time when the blood flow becomes minimum amount, or the time when the blood flow becomes maximum amount.

FIG. 13 shows an example, which a time when a blood flow becomes minimum amount is detected automatically, and a marker M1 is displayed at the automatically detected time when the blood flow becomes the minimum amount. Thus, the marker M1 may be displayed on a graph by making image information for displaying the marker M1 on the display unit 12 in the cardiac output calculation unit 10.

Next, using the detected time in the cardiac output calculation unit 10 checks the periodicity. That is, between times when stable period is obtained, i.e., between a certain time when the blood flow becomes the minimum amount and a time when the blood flow becomes the minimum amount next, is chosen as a section for one cycle. The blood flow during the time chosen as the section for one cycle comprises each instantaneous flow of every time Tv corresponding to 1 Volume, as shown in FIG. 14. Then, the cardiac output calculation unit 10 calculates a cardiac output by integrating each instantaneous blood flow in the section for one selected cycle.

There is an advantage of being difficult to be influenced by posture movement of an object in such a method of detecting one cardiac cycle based on an amount change of a blood flow compared with a method of detecting one cardiac cycle synchronizing with an ECG signal of an electrocardiogram.

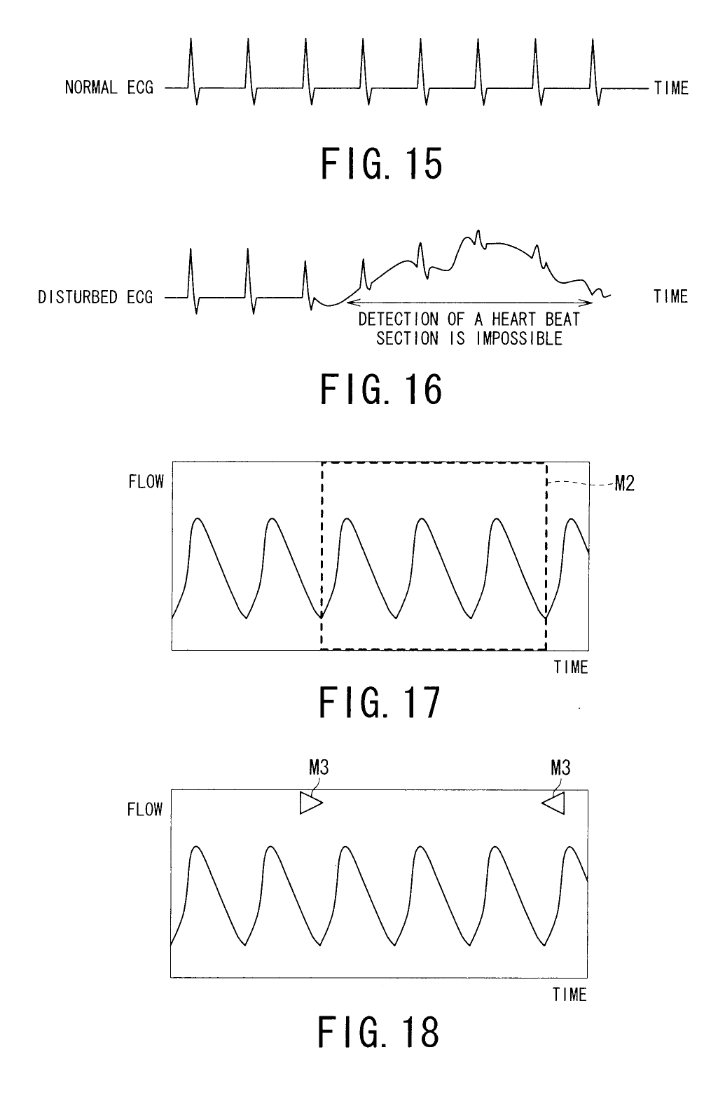

FIG. 15 is an electrocardiogram showing a normal stable ECG waveform normally used for detection of one cardiac cycle. FIG. 16 is an example of electrocardiogram showing a disturbed ECG waveform due to changing a body position of object.

In FIGS. 15 and 16, each abscissa denotes time and each ordinate denotes a value of an ECG signal. A method of detecting one cardiac cycle from an electrocardiogram presenting a normal stable ECG waveform as shown in FIG. 15 is often taken. However, if posture changes due to an object's movement, an ECG waveform will be disturbed as shown in FIG. 16. For this reason, there is a possibility that one cardiac cycle may not be appropriately detected in response to the influence of the posture movement of the object, when one cardiac cycle is going to be detected from the ECG waveform as shown in FIG. 16.

On the other hand, if one cardiac cycle is detected based on an amount change of a blood flow, the influence by posture movement of the object will be suppressed and detecting one cardiac cycle appropriately will become possible.

A cardiac output is also computable by a method other than a method of calculating a cardiac output by integrating each instantaneous blood flow in a section of one cardiac cycle as mentioned above. For example, a more stable cardiac output can be calculated if an average regarding heart beats is calculated by dividing a sum obtained by adding instantaneous blood flows included in a section corresponding to a plurality of periods with the number of heart beat included in the section for the addition target.

The cardiac output obtained like this is provided from the cardiac output calculation unit 10 to the display unit 12 and displayed thereon. For this reason, an operator can notice the cardiac output of the object as a numerical value by viewing the display unit 12 of the ultrasonic diagnostic apparatus 1. A color Doppler image and the graph of the time change of the blood flow displayed with the cardiac output are always updated with a progress of time required for image generation for 1 Volume. Then, the cardiac output, which is to be displayed on the display unit 12 as a numerical value, may always be updated by calculating the cardiac output for every heart beat, for example.

When a cardiac output is calculated by using an average regarding heart beats from instantaneous blood flows included in a section corresponding to a plurality of periods, displaying a range of the instantaneous blood flows used for the calculation of the cardiac output, on the display unit 12 may be useful for diagnosis. Then, it is also possible to display the range of the instantaneous blood flows used for calculating the cardiac output visually on the display unit 12 by using a sign and/or a color. Image information for displaying the range of the instantaneous blood flows visually can be made in the cardiac output calculation unit 10, and can be outputted to the display unit 12.

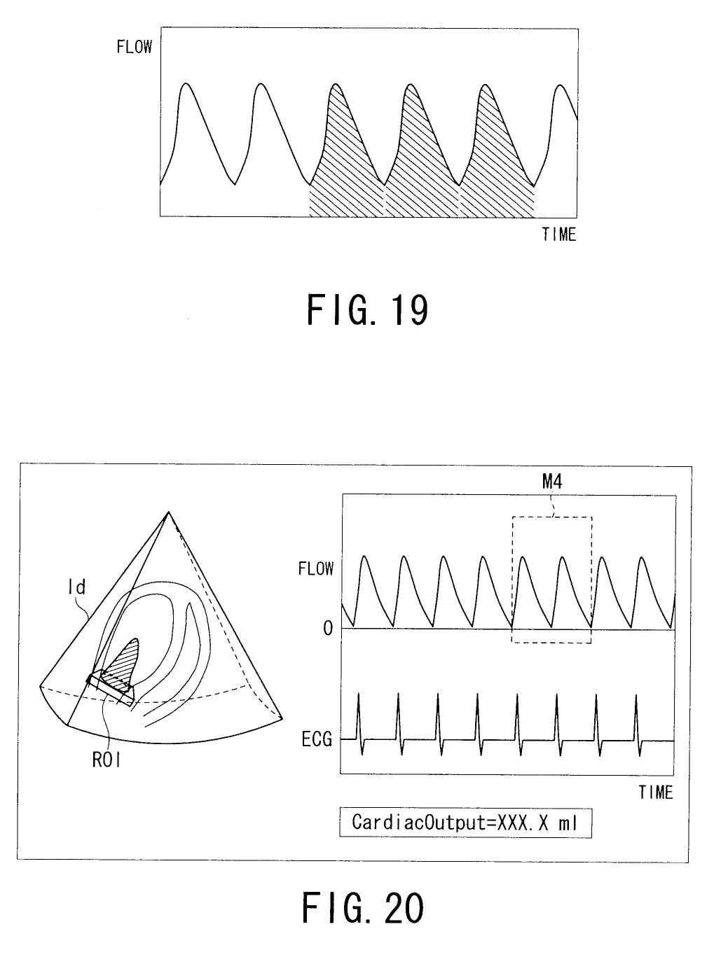

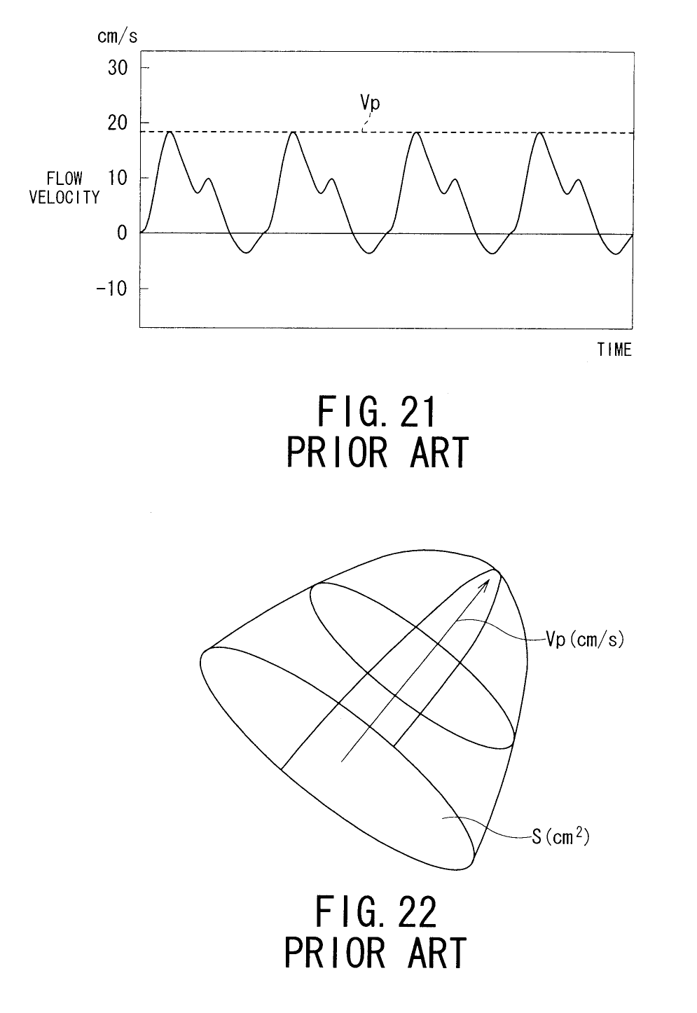

FIG. 17 is a diagram showing an example of display of a region for instantaneous blood flow used for calculating a cardiac output with using a marker shown by a dotted line on the display unit 12 shown in FIG. 1. FIG. 18 is a diagram showing an example of display of a region for instantaneous blood flow used for calculating a cardiac output with using markers each shown by a triangular symbol on the display unit 12 shown in FIG. 1. FIG. 19 is a diagram showing an example of display of a region for instantaneous blood flow used for calculating a cardiac output by color or pattern indication on the display unit 12 shown in FIG. 1.

In FIGS. 17, 18 and 19, each abscissa denotes time and each ordinate denotes a blood flow (quantity). A section of the instantaneous blood flows used for calculating the cardiac output can be display on the display unit 12 visually with a marker M2 shown by a dotted line, as shown in FIG. 17. Alternatively, a section of the instantaneous blood flows used for calculating the cardiac output may also be displayed on the display unit 12 visually with two markers M3 shown by triangular signs, as shown in FIG. 18. Further, a section of the instantaneous blood flows used for calculating the cardiac output may also be displayed on the display unit 12 visually by coloring or patterning the section, as shown in FIG. 19.

Furthermore, the three-dimensional color Doppler image, the time change graph of the blood flow and the cardiac output obtained as mentioned above can be displayed in parallel on the display unit 12.

FIG. 20 is a diagram showing an example of window in case of displaying a three-dimensional color Doppler image, a graph indicating a temporal variation of blood flow and a cardiac output together on the display unit 12 of the ultrasonic diagnostic apparatus 1 shown in FIG. 1.

The color Doppler image Id is displayed with a three-dimensional scan range on the left-hand side of the display unit 12 as shown in FIG. 20. A B mode image prepared separately may also be superpositioned on the color Doppler image Id for displaying. A ROI is set on the color Doppler image Id as a calculation range of the blood flow. Further, the time change graph of the blood flow on a certain section set as the ROI is displayed on the right-hand side of the display unit 12. Furthermore, a graph presenting a time change of a bio signal such as an electrocardiogram may also be displayed in synchronized with the time change graph of the blood flow in time.

Moreover, a numerical indication of the cardiac output is displayed at the lower part of the display unit 12. Further, the range of the instantaneous blood flows used for calculating the cardiac output is visually shown on the time change graph of the blood flow with the marker M4 shown by a dotted line. For this reason, an operator may easily notice the amount of blood flow and the cardiac output together with the color Doppler image Id in real time or afterwards by referring the display unit 12.

That is, the above mentioned ultrasonic diagnostic apparatus 1 is to obtain time change of blood flow using color Doppler velocity information acquired by a three-dimensional scan and to display the obtained time change of the blood flow visually as a graph. Time which is the abscissa of the graph displayed on the ultrasonic diagnostic apparatus 1 correlates with a scan position.

For this reason, according to the ultrasonic diagnostic apparatus 1, a history of flow change due to movement of a scan position can be referred simply without depending on memory of an operator. Further, a scan position where a blood flow becomes the largest can be found in a short time if a scan is carried out with referring a time change graph of a blood flow, or if a time change graph of a blood flow is referred after a scan. For this reason, it becomes possible to perform a stable scan in a scan position where the largest blood flow can be measured.

Further, according to the ultrasonic diagnostic apparatus 1, a cardiac output can be calculated based on a time change of a blood flow. For this reason, the influence of motions due to postural movement and breathing of an object can be suppressed compared with a case where a cardiac output is calculated based on an electrocardiogram. Therefore, measuring an accurate blood flow in a short period of time becomes possible. Further, improving the diagnosis efficiency and lightening an operator's burden can also be achieved. Specifically, diagnostic efficiency of heart function and the reliability indicated as a value of measurement accuracy of a cardiac output or the like can be raised remarkably.

Although the embodiment mentioned above shows an example in which a blood flow is immediately calculated from color Doppler velocity information acquired by a scan with transmitting and receiving an ultrasonic wave and a time change graph of the blood flow is displayed immediately, i.e. the so-called real time processing is performed, each processing can be performed not only in real time but also at an arbitrary timing. For example, calculation of a blood flow based on color Doppler velocity information and displaying a time change graph of the blood flow may be performed respectively at an arbitrary timing after an ultrasonic scan.

As a more specific example, a case may be mentioned that color Doppler images for a predetermined time is memorized in a cine image memory or saved in an HDD (hard disk drive) after acquiring color Doppler velocity information by an ultrasonic scan. Usually, a displaying time for a plurality of color Doppler images covering a few of heart beats is about 2 seconds to 30 seconds, and the color Doppler images are often memorized in a cine image memory or saved in an HDD. In such a case, necessary color Doppler images can be read out at a desired opportunity after the preservation of the color Doppler images, and then a spatial region where a blood flows is requested to be measured can be set as a ROI. Further, an instantaneous blood flow can be calculated by integration under a method similar to that in the case of real time processing mentioned above, using the color Doppler velocity information in the spatial region set as the ROI. Furthermore, if instantaneous blood flows in ROIs for a plurality of color Doppler images is similarly calculated, time change graphs of blood flows can be generated based on the respective instantaneous blood flows corresponding to the color Doppler images respectively.

Moreover, it was explained in the embodiment mentioned above that data groups of color Doppler velocity information acquired by a three-dimensional scan ware pieces of Volume data arranged at equal intervals. However, data groups of color Doppler velocity information are not necessarily needed to be pieces of Volume data arranged at equal intervals. It is possible to generate a similar time change graph of a blood flow even if a data interval in arbitrary directions differs from that in another direction among a lengthwise direction, a transverse direction, and a depth direction toward a color Doppler image.

Furthermore, although calculating a blood flow in a heart with setting a ROI in the heart was explained as an example, a ROI may be set not only in a heart but in an arbitrary part. Then, a flow in a blood vessel in an arbitrary part other than a heart may be calculated and represented graphically.

What is claimed is:

1. An ultrasonic diagnostic apparatus comprising:
a Doppler velocity information acquiring unit configured to acquire three-dimensional Doppler velocity information from an object by a three-dimensional scan with transmission and reception of an ultrasonic wave;
a region-of-interest-setting unit configured to set a region of interest spatially; and
an instantaneous flow calculating unit configured to calculate an instantaneous blood flow in the region of interest with using the three-dimensional Doppler velocity information.

2. An ultrasonic diagnostic apparatus according to claim 1,
wherein said region-of-interest-setting unit is configured to set the region of interest as a two-dimensional area.

3. An ultrasonic diagnostic apparatus according to claim 1, further comprising
a graph display unit configured to generate graph information for indicating a temporal variation of a blood flow based on the instantaneous blood flow and display a graph showing the temporal variation of the blood flow according to the graph information.

4. An ultrasonic diagnostic apparatus according to claim 3,
wherein said graph display unit is configured to generate the graph information so as to display a temporal variation of a living body signal in synchronized with the temporal variation of the blood flow in time phase.

5. An ultrasonic diagnostic apparatus according to claim 3,
wherein said graph display unit is configured to display the graph together with a Doppler image obtained from the three-dimensional Doppler velocity information in real time.

6. An ultrasonic diagnostic apparatus according to claim 3,
wherein said graph display unit is configured to display the graph at a designated timing after the three-dimensional scan.

7. An ultrasonic diagnostic apparatus according to claim 1,
wherein said instantaneous flow calculating unit is configured to calculate short three-dimensional Doppler velocity information for calculating the instantaneous blood flow by interpolation with using other acquired three-dimensional Doppler velocity information.

8. An ultrasonic diagnostic apparatus according to claim 1,
wherein said instantaneous flow calculating unit is configured to calculate short three-dimensional Doppler velocity information for calculating the instantaneous blood flow by interpolation with using other acquired three-dimensional Doppler velocity information and determine a scan section so as to obtain a necessary interpolation accuracy.

9. An ultrasonic diagnostic apparatus according to claim 3, further comprising
a cardiac output calculating unit configured to calculate a blood flow in one heart rate based on the graph information.

10. An ultrasonic diagnostic apparatus according to claim 3, further comprising
a cardiac output calculating unit configured to calculate a blood flow in one heart rate by detecting an interval corresponding to one period of a blood flow varying periodically from the graph information and integrating the instantaneous blood flow in the detected interval.

11. An ultrasonic diagnostic apparatus according to claim 3, further comprising
a cardiac output calculating unit configured to calculate a blood flow in one heart rate by detecting an interval corresponding to plural periods of a blood flow varying periodically from the graph information and performing average processing with using the number of the detected plural periods to an integral value of the instantaneous blood flow in the detected interval.

12. An ultrasonic diagnostic method comprising steps of:
acquiring three-dimensional Doppler velocity information from an object by a three-dimensional scan with transmission and reception of an ultrasonic wave;
setting a region of interest spatially; and
calculating an instantaneous blood flow in the region of interest with using the three-dimensional Doppler velocity information.
